(19) United States
(12) Patent Application Publication
(10) Pub. No.: US 2025/0349004 A1
(43) Pub. Date: Nov. 13, 2025

(54) Systems and Methods for Anatomical Shape Modeling

(71) Applicant: The Board of Trustees of the Leland Stanford Junior University, Stanford, CA (US)

(72) Inventors: Anthony Gatti, Stanford, CA (US); Akshay Chaudhari, Mountain View, CA (US)

(73) Assignee: The Board of Trustees of the Leland Stanford Junior University, Stanford, CA (US)

(21) Appl. No.: 19/201,860

(22) Filed: May 7, 2025

Related U.S. Application Data
(60) Provisional application No. 63/643,867, filed on May 7, 2024.

(51) Int. Cl. G06T 7/00; G06T 7/11; G16H 50/20

(52) U.S. Cl. CPC G06T 7/0012; G06T 7/11; G16H 50/20; G06T 2207/10088; G06T 2207/20084; G06T 2207/30008

(57) ABSTRACT

Systems and methods for anatomical shape modeling in accordance with embodiments of the invention are illustrated. One embodiment includes a method for clinical anatomy modeling, comprising obtaining imaging data of a portion of a patient's anatomy, generating a first model of the patient's anatomy based on the imaging data, providing the first model to a trained hybrid explicit-implicit neural shape model (NSM), obtaining a latent representation of the first model from the trained hybrid explicit-implicit NSM, and reconstructing a second model of the portion of the patient's anatomy using the latent representation. In a further embodiment, the second model is at a higher resolution than the first model. In a yet further embodiment, the method further includes steps for providing the latent representation to a classification model trained to classify latent representations to clinical values.

### CROSS-REFERENCE TO RELATED APPLICATIONS

The current application claims the benefit of and priority under 35 U.S.C. § 119(e) to U.S. Provisional Patent Application No. 63/643,867 entitled “Neural Shape Model to Encode Anatomic Shape and Quantify Disease” filed May 7, 2024. The disclosure of U.S. Provisional Patent Application No. 63/643,867 is hereby incorporated by reference in its entirety for all purposes.

### FIELD OF THE INVENTION

The present invention generally relates to automated generation of high quality 3D reconstructions of anatomical image data and disease state prediction.

### BACKGROUND

Radiology is a medical field concerned with non-invasively imaging the internal structures of the body. There are a number of different medical imaging modalities with various benefits and detriments. Magnetic resonance imaging (MRI) is a non-invasive medical imaging technique that uses strong magnetic fields and radio waves to generate detailed images of internal body structures. Computed tomography (CT) is another widely used medical imaging modality that employs X-rays to create cross-sectional images of the body. CT scanners rotate around the patient, taking multiple X-ray images from different angles, which are then reconstructed into 3D representations of internal structures.

Machine learning is a field of computer science concerned with training models to perform tasks based on various inputs. There are a wide number of different types of machine learning models that have been developed and specialized for different tasks. A common class of machine learning model is the neural network, where a group of nodes are connected, where each node receives a signal, processes it, and passes on the result to connected neurons via edges. The processing component of each node can be trained to produce superior results during a training phase, where labeled training data is typically used to train the parameters of nodes to minimize a loss function. Different network architectures have been generated for particular tasks. For example, convolutional neural networks (CNNs) are particularly suited for classifying image data. Autoencoders are particularly adept at learning efficient coding of unlabeled data, rather than labeled data typically used in a supervised learning environment.

### SUMMARY OF THE INVENTION

Systems and methods for anatomical shape modeling in accordance with embodiments of the invention are illustrated. One embodiment includes a method for clinical anatomy modeling, comprising obtaining imaging data of a portion of a patient's anatomy, generating a first model of the patient's anatomy based on the imaging data, providing the first model to a trained hybrid explicit-implicit neural shape model (NSM), obtaining a latent representation of the first model from the trained hybrid explicit-implicit NSM, and reconstructing a second model of the portion of the patient's anatomy using the latent representation.

In a further embodiment, the second model is at a higher resolution than the first model.

In still another embodiment, generating the first model includes segmenting the imaging data, and fitting a mesh to the segmented image data.

In a still further embodiment, the hybrid explicit-implicit neural shape model comprises an explicit portion, comprising a dense layer, a reshaping layer, and a convolutional neural network trained to output triplanar features, and an implicit portion, comprising a multilayer perceptron trained to convert a local latent representation based on triplanar features into a signed distance between two surfaces.

In yet another embodiment, the obtained latent representation is an implicit latent representation from the implicit portion of the hybrid explicit-implicit neural shape model.

In a yet further embodiment, the method further includes steps for providing the latent representation to a classification model trained to classify latent representations to clinical values.

In another additional embodiment, the clinical values are magnetic resonance imaging osteoarthritis knee scores.

In a further additional embodiment, the imaging data is magnetic resonance imaging data.

In another embodiment again, the portion of the patient's anatomy includes the patient's knee and femur.

One embodiment includes a clinical anatomy modeling system, comprising a processor, and a memory, the memory containing a modeling application that directs the processor to obtain imaging data of a portion of a patient's anatomy generated by an imaging device, generate a first model of the patient's anatomy based on the imaging data, provide the first model to a trained hybrid explicit-implicit neural shape model (NSM), obtain a latent representation of the first model from the trained hybrid explicit-implicit NSM, and reconstruct a second model of the portion of the patient's anatomy using the latent representation.

In a further embodiment again, the second model is at a higher resolution than the first model.

In still yet another embodiment, to generate the first model, the modeling application further directs the processor to segment the imaging data, and fitting a mesh to the segmented image data.

In a still yet further embodiment, the hybrid explicit-implicit neural shape model comprises an explicit portion, comprising a dense layer, a reshaping layer, and a convolutional neural network trained to output triplanar features, and an implicit portion, comprising a multilayer perceptron trained to convert a local latent representation based on triplanar features into a signed distance between two surfaces.

In still another additional embodiment, the obtained latent representation is an implicit latent representation from the implicit portion of the hybrid explicit-implicit neural shape model.

In a still further additional embodiment, the modeling application further directs the processor to provide the latent representation to a classification model trained to classify latent representations to clinical values.

In still another embodiment again, the clinical values are magnetic resonance imaging osteoarthritis knee scores.

In a still further embodiment again, the imaging data is magnetic resonance imaging data.

In yet another additional embodiment, the portion of the patient's anatomy includes the patient's knee and femur.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates an automated anatomical modeling system in accordance with an embodiment of the invention.

FIG. 2 is a block diagram for a modeling device in accordance with an embodiment of the invention.

FIG. 3 is a flow chart for a training process for shape models and classification models in accordance with an embodiment of the invention.

FIG. 4 is a set of loss functions for training neural shape models in accordance with an embodiment of the invention.

FIG. 5 is a graphical representation of a hybrid explicit implicit neural shape model in accordance with an embodiment of the invention.

FIG. 6 is a flow chart for an enhancement process in accordance with an embodiment of the invention.

### DETAILED DESCRIPTION

The analysis of anatomical structures plays a crucial role in medical diagnosis, treatment planning, and research. While MRI and CT scans have enabled 3D reconstruction of internal body parts, there remain several issues. First, depending on the quality of the machine, the quality of the output may not be high. For example, lower Tesla MRI machines produce lower resolution images. Second, the imaging data produced by the imaging device still needs to be interpreted by a trained radiologist. Systems and methods described herein enable significant enhancement of 3D image data using shape models which can result in higher resolution 3D models compared to the typical output of a given medical imaging device. These 3D models can be used for surgical planning, structural analysis, and any other medical purpose as appropriate to the requirements of specific applications of embodiments of the invention. Secondly, the latent representation produced by the shape model can be used as input to a diagnostic model trained to automatically identify disease conditions or abnormalities in the imaged patient based on the imaged anatomy. For example, radiographic scores can be automatically generated, or even specific diseases can be identified depending on the particular training process and model used.

By way of specific example, osteoarthritis (OA) is the leading cause of pain and disability in developed countries. OA affects all tissues in a joint, with emphasis on bone and cartilage. The most OA research focuses on 2D CNNs applied to X-rays, 2D and 3D CNNs for segmentation and classification of MRI images. Characterizing OA relies on medical imaging to discern the shape of anatomic tissues. As OA progresses, osteophytes grow at the edges of cartilage, and the cartilage is thinned. Diagnosis depends on these anatomical changes. Anatomical shape diagnoses are critical in other conditions such as, but not limited to, craniosynostosis and numerous orthopedic conditions.

Shape modeling provides an avenue for identifying these anatomical symptoms. However, current shape models are limited in their usefulness. Statistical shape models (SSMs) require anatomic point matching, which is not guaranteed to be possible for every patient and indeed may not be possible for certain diseases. Osteophytes that form in OA are not present in healthy bones, and therefore no true matching point exists. While SSMs are useful in identifying gross features or predicting disease in general, accurate quantification of specific, localized biomarkers are needed for clinical applications. Neural shape models (NSMs) are generative shape models in the medical domain, however these shape models generate implicit representations of the object. Described herein is a hybrid explicit-implicit neural shape model that outperforms SSMs and implicit NSMs for bone and cartilage reconstruction, and SSMs, implicit NSMs, and CNNs in disease classification tasks.

Hybrid explicit-implicit NSMs utilize a CNN to generate an explicit representation, and a multi-layer perceptron (MLP) with ReLU activations is then used to generate an implicit representation of the anatomy. The implicit representations produced by hybrid explicit-implicit NSMs outperform previous implicit only NSMs and SSMs. An advantage of this architecture is interpretability. NSMs are generative, and therefore it can be observed whether the model is capturing features of interest for a given patient. This can provide a significant backstop against false positives and false negatives in disease classification. Further, by modifying the latent space to add or remove features, an in silico twin of a given patient can be generated for clinical use.

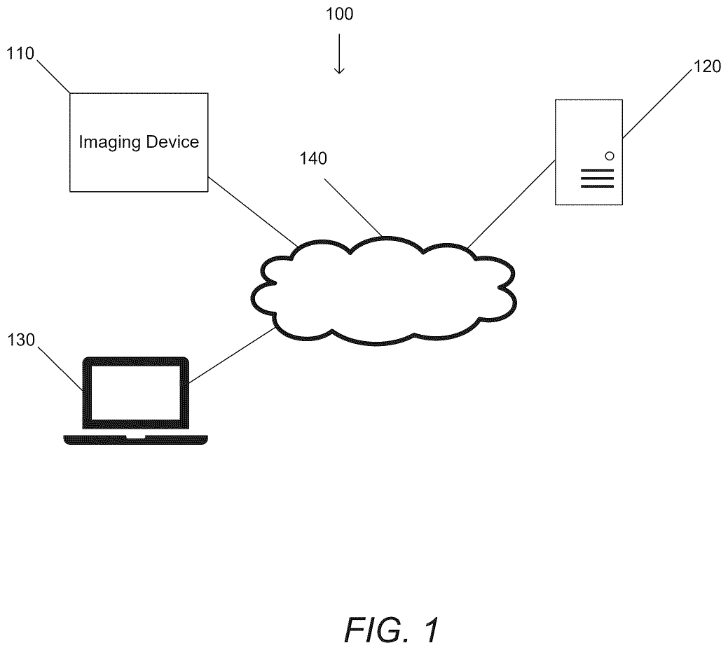

Turning now to FIG. 1, a system for anatomical modeling in accordance with an embodiment of the invention is illustrated. Anatomical modeling system 100 includes an imaging device 110. In many embodiments, the imaging device is an MRI or a CT scanner. However other imaging modalities capable of providing 3D shape information can be used. For example, medical computer vision systems that generate models of surface anatomical features can be used. A modeling device 120 is included in system 100. Modeling devices are computational platforms capable of carrying out anatomical modeling processes as described herein. Outputs of modeling devices can be visualized using display device 130. As shown in FIG. 1, different devices can be connected via a network 140, such as (but not limited to) the Internet, an intranet, or a combination of multiple networks. In some embodiments, the components are not all directly connected but are capable of receiving data produced by certain other devices in the system.

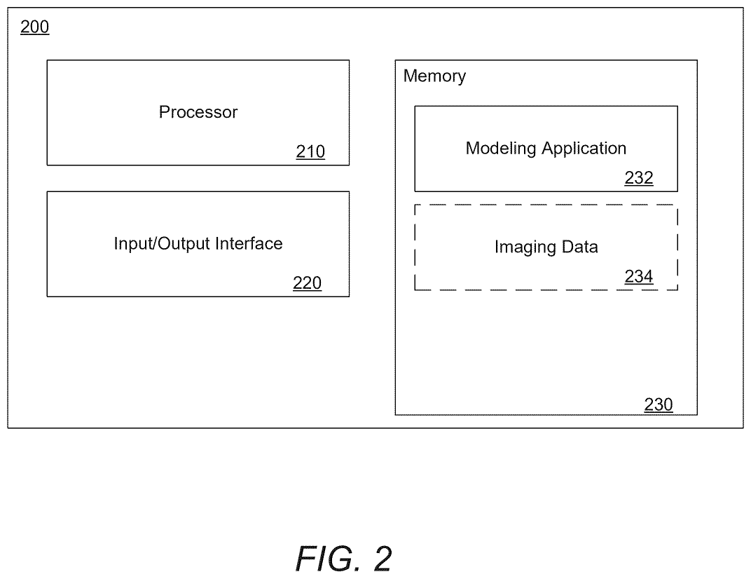

Turning now to FIG. 2, a block diagram for a modeling device in accordance with an embodiment of the invention is illustrated. Modeling device 200 includes a processor 210. In many embodiments, processors are any logic circuitry capable of performing anatomical modeling processes described herein. For example, logic circuitry can include (but is not limited to) central processing units (CPUs), graphics processing units (GPUs), field-programmable gate arrays (FPGAs), application-specific integrated circuits (ASICs), and/or any other computing circuit or combination thereof as appropriate to the requirements of specific applications of embodiments of the invention.

Modeling device 200 further includes an input/output (I/O) interface 220. I/O interfaces are capable of sending and receiving information to and from other connected devices. In many embodiments, I/O interfaces contain multiple I/O modalities, i.e. wired and/or wireless communication ports. Modeling device 200 further includes a memory 230. Memory can be volatile memory, non-volatile memory, or a combination thereof. The memory 230 stores a modeling application 232 which contains a set of instructions that configures the processor 210 to perform various processes described herein. At various points during operation, the memory may also store imaging data 234 of a patient generated by an imaging device. While a particular system architecture and modeling device architecture are illustrated in FIGS. 1 and 2, as can be readily appreciated, any number of different architectures can be used without departing from the scope or spirit of the invention.

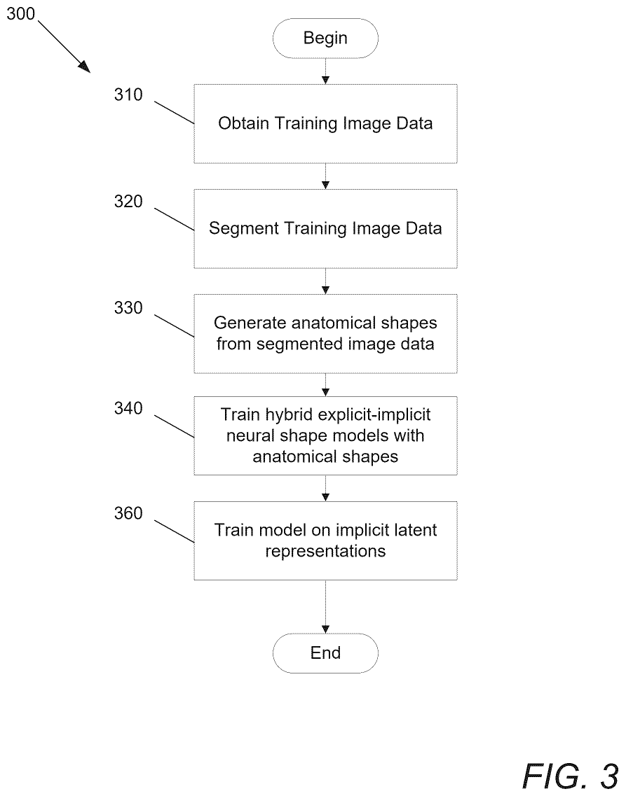

Turning now to FIG. 3, a process for training a hybrid explicit-implicit NSM and a subsequent model in accordance with an embodiment of the invention is illustrated. Process 300 includes obtaining (310) a training image data set. In many embodiments, the training image data set contains image data generated by a particular modality, e.g. MRI or CT. The training image data describes many 3D representations of the same anatomic structure, but from many different individuals at various points in time. The training image data set is segmented (320) to extract just the anatomical features of interest from the surrounding void (or other tissue). A 3D model of each anatomy from each image in the segmented image data is generated (330) based on the segmented training image data. In many embodiments, a mesh is applied to the segmented training image data to create the mesh. However, other methods such as (but not limited to) point-cloud based methods can be used as appropriate to the requirements of specific applications of embodiments of the invention. A hybrid explicit-implicit NSM can then be trained (340) by fitting the model to the set of generated 3D models.

In furtherance of the example of OA, during the generation of the training 3D models, an arbitrary mesh can be chosen as the reference. Every other bone mesh can be registered to the reference using a similarity transform (rigid+scale), which is applied to the coinciding cartilage surface. Next, bone and cartilage meshes can be centered using the mean of the bone points and were normalized using maximum radial distance so both tissues lie within a unit sphere. Then, separately for the bone and cartilage surfaces, 500,000 points were sampled. Approximately ninety percent of points can be randomly sampled by first sampling positions on the surface using blue noise to produce uniform random samples. Then, sampled surface points can be perturbed by adding zero mean Gaussian noise: 45% σ=0.016; 45% σ=0.05. The remaining 10% of points can be uniformly sampled over the unit cube. Finally, the signed distance s from both meshes can be calculated for every sampled point.

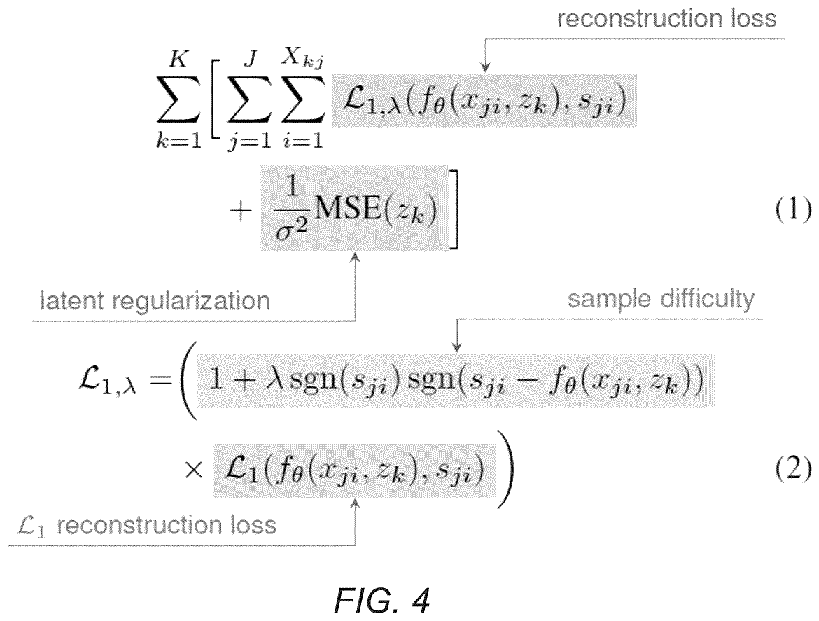

Prior to training, each bone/cartilage pair can be assigned a random z˜ [symbol] (0, 0.01²). During training, for each subject (k) and surface type (j: bone/cartilage), 17,000 points (Xⱼₖ) are randomly sampled with equal numbers of points inside (−) and outside (+) the surface. A reconstruction loss function with latent regularization as seen in FIG. 4, equation (1) is optimized to minimize the error in predicted s and to regularize the latent z. The loss comprises a reconstruction and latent regularization term, where the reconstruction term penalizes hard samples (predicted wrong sign) as shown in equation (2) of FIG. 4, and includes a weighted [symbol] ₁ where λ(0,1) controls the weighting on hard samples with λ=0 being equivalent to regular [symbol] ₁ and higher values provide greater penalty. ∧ can be exponentially increased from 0 to 0.2 over the first approximately 1800 epochs. A latent regularization loss independently penalizes each z component with σ=100 to promote diagonal covariance. Latents and network weights f_{θ} can be jointly optimized using the AdamW optimizer with weight decay of 1e-4. As can be readily appreciated, while a specific training data preprocessing steps and training steps are enumerated with respect to the OA use case, similar steps and/or different steps can be applied to different use cases that utilize hybrid explicit-implicit NSMs without departing from the scope or spirit of the invention.

A variant of the hybrid explicit-implicit NSM itself can be used to reconstruct shape-specific latents. In many embodiments, the trained hybrid explicit-implicit NSM weights are frozen and then the model is fit to the new surfaces. For example, the bone to be reconstructed is similarly registered to the mean bone shape of the NSM (zero-vector) and the bone/cartilage surfaces are scaled to be within a unit sphere. Then, a randomly initialized latent z˜ [symbol] (0, 0.01²) is optimized for ˜2,000 epochs to reconstruct the surfaces using an [symbol] ₁ loss between the network predicted signed distance s and the actual s of ˜20,000 randomly sampled surface points (s=0) using an optimizer. The Ir can be decayed by a factor of 0.9 every 20 epochs, and early stopping can be implemented with a patience of approximately 50 epochs.

Again with respect to FIG. 3, the trained hybrid explicit-implicit NSM generates an explicit and an implicit representation of the 3D model. One or more of the representations can be extracted and used to train (350) a separate model to perform any of a number of tasks such as (but not limited to) model visualization, disease classification, or any other trainable task as appropriate to the requirements of specific applications of embodiments of the invention. In many embodiments, the implicit representation from the hybrid explicit-implicit NSM is used to train the subsequent model. However, the explicit and/or both the explicit and implicit representations can be used as appropriate to the requirements of specific applications of embodiments of the invention. In many embodiments, the training process for the subsequent model includes providing the latent representation of a 3D model along with a labeling, where the training process trains the subsequent model to apply the appropriate label for a given provided 3D model. As a specific example, for OA, the labeling may be MRI Osteoarthritis Knee Score (MOAKS), OA staging, OA diagnosis, future disease prediction, or the likelihood of requiring a knee replacement surgery. As can be readily appreciated, the labeling can be anything, but preferably is a clinically relevant classification of the particular anatomy.

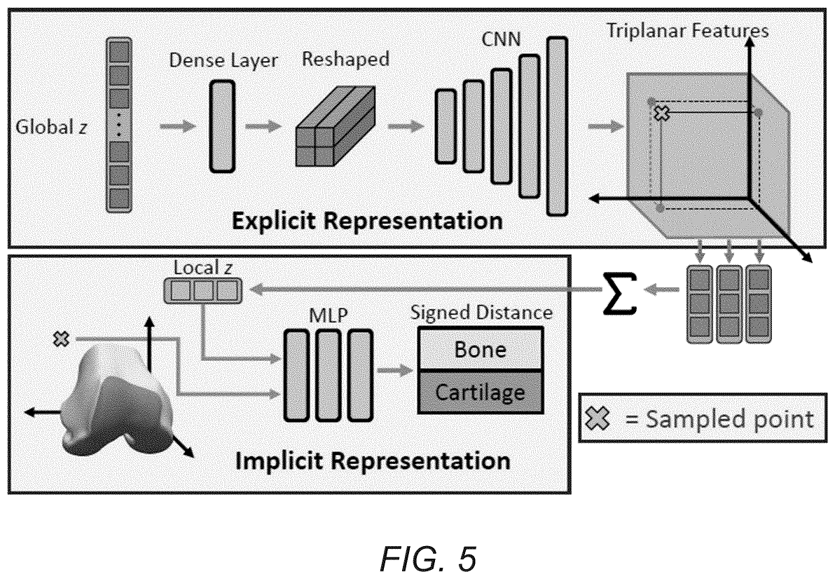

Turning now to FIG. 5, a graphical representation of an example hybrid explicit-implicit NSM architecture in accordance with an embodiment of the invention is illustrated. The hybrid explicit-implicit NSM has two main components: the explicit representation and the implicit representation. A global latent z controls the overall generated shape. The global z is passed through a dense layer, reshaped and then fed through a 5-layer CNN to produce 64×64 2D outputs with 384 feature maps. The 384 feature maps are split into 3 to produce one set of 64×64×128 feature maps per orthogonal plane. To determine the signed distance of a particular point \textcircled{x}, that point is projected onto each feature map plane and the corresponding feature vector is extracted using bilinear interpolation. These plane-specific feature maps are summed, yielding the local z. The local z is a coordinate-specific latent vector that controls the signed distance prediction. The local z along with the XYZ coordinates of point \textcircled{x} are passed to a three-layer multilayer perceptron (MLP) which outputs the signed distance of two surfaces (e.g. bone and cartilage).

In many embodiments, a global latent z of a length of 512 is processed via a fully connected layer, resulting in a 2048-length vector. This vector is then reshaped to be 2×2×512 before being input into the CNN decoder. The CNN decoder has 5 2D transpose convolution layers, with stride 2 and 512 channels as outputs at each layer. The final output layer of the CNN is sized 64×64×384; the 384 features maps were split into 128 features per orthogonal plane. Sampled points x∈ [symbol] ³ are projected onto the three orthogonal planes, and a length 128 z was obtained per feature plane via the bilinear interpolation. Plane features can be combined via summation, yielding a length 128 local z. The local z and the sampled x position can be concatenated and input into the implicit 3-layer MLP with width 512, ReLU activations, and a length two output (one for each tissue) with a tanh activation.

In many embodiments, the implicit decoder is an 8-layer MLP of width 512, with a skip connection of the inputs (x and z) to layer 4, and ReLU activations throughout. The output is sized two and uses tanh activation. While specific layer numbers, sizes, and cardinalities are discussed above explicit and implicit portions with respect to specific implementations, these can be modified as appropriate to the requirements of specific applications of embodiments of the invention.

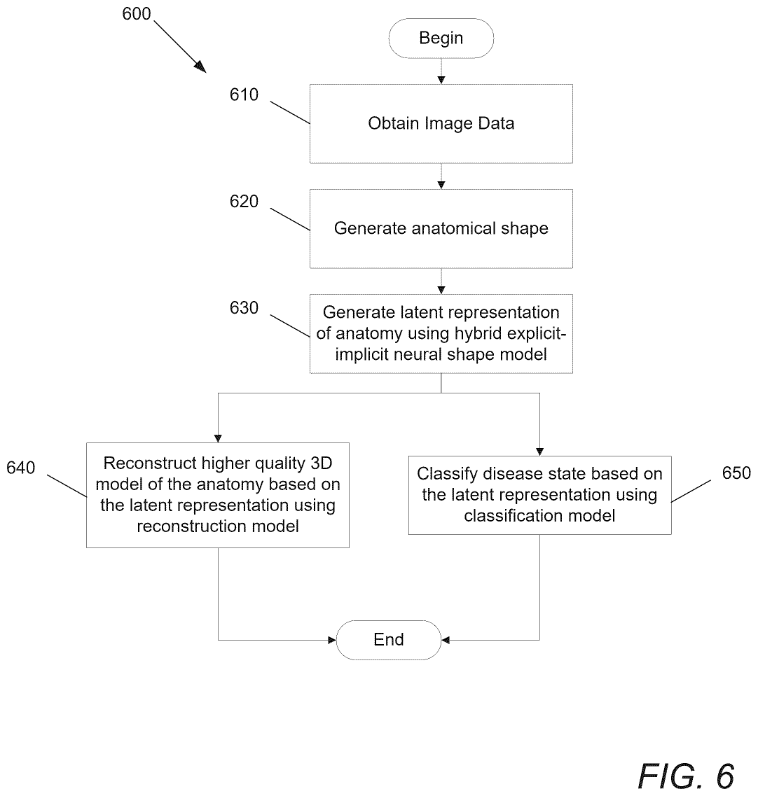

Turning now to FIG. 6, a process for utilizing trained hybrid explicit-implicit NSMs in accordance with an embodiment of the invention is illustrated. Process 600 includes obtaining (610) image data of a patient describing a portion of the patient's anatomy. The image data is segmented to generate (620) an anatomical shape model similar to the training process above. The trained hybrid explicit-implicit NSM is provided with the anatomical shape model derived from the image data and generates (630) a latent representation of the anatomy. The latent representation can be used to reconstruct (640) a higher quality 3D model of the anatomy using a reconstruction model, and/or used to classify (650) a disease state using a classification model.

Although specifics are discussed above, many different methods and variants of the machine learning model architectures can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for clinical anatomy modeling, comprising:
obtaining imaging data of a portion of a patient's anatomy;
generating a first model of the patient's anatomy based on the imaging data;
providing the first model to a trained hybrid explicit-implicit neural shape model (NSM);
obtaining a latent representation of the first model from the trained hybrid explicit-implicit NSM; and
reconstructing a second model of the portion of the patient's anatomy using the latent representation.

2. The method of claim 1, wherein the second model is at a higher resolution than the first model.

3. The method of claim 1, wherein generating the first model comprises segmenting the imaging data, and fitting a mesh to the segmented image data.

4. The method of claim 1, wherein the hybrid explicit-implicit neural shape model comprises:
an explicit portion, comprising:
a dense layer;
a reshaping layer; and
a convolutional neural network trained to output triplanar features; and
an implicit portion, comprising:
a multilayer perceptron trained to convert a local latent representation based on triplanar features into a signed distance between two surfaces.

5. The method of claim 1, wherein the obtained latent representation is an implicit latent representation from the implicit portion of the hybrid explicit-implicit neural shape model.

6. The method of claim 1, further comprising providing the latent representation to a classification model trained to classify latent representations to clinical values.

7. The method of claim 6, wherein the clinical values are magnetic resonance imaging osteoarthritis knee scores.

8. The method of claim 1, wherein the imaging data is magnetic resonance imaging data.

9. The method of claim 1, wherein the portion of the patient's anatomy comprises the patient's knee and femur.

10. A clinical anatomy modeling system, comprising:
a processor; and
a memory, the memory containing a modeling application that directs the processor to:
obtain imaging data of a portion of a patient's anatomy generated by an imaging device;
generate a first model of the patient's anatomy based on the imaging data;
provide the first model to a trained hybrid explicit-implicit neural shape model (NSM);
obtain a latent representation of the first model from the trained hybrid explicit-implicit NSM; and
reconstruct a second model of the portion of the patient's anatomy using the latent representation.

11. The system of claim 10, wherein the second model is at a higher resolution than the first model.

12. The system of claim 10, wherein to generate the first model, the modeling application further directs the processor to segment the imaging data, and fitting a mesh to the segmented image data.

13. The system of claim 10, wherein the hybrid explicit-implicit neural shape model comprises:
an explicit portion, comprising:
a dense layer;
a reshaping layer; and
a convolutional neural network trained to output triplanar features; and
an implicit portion, comprising:
a multilayer perceptron trained to convert a local latent representation based on triplanar features into a signed distance between two surfaces.

14. The system of claim 10, wherein the obtained latent representation is an implicit latent representation from the implicit portion of the hybrid explicit-implicit neural shape model.

15. The system of claim 10, wherein the modeling application further directs the processor to provide the latent representation to a classification model trained to classify latent representations to clinical values.

16. The system of claim 15, wherein the clinical values are magnetic resonance imaging osteoarthritis knee scores.

17. The system of claim 10, wherein the imaging data is magnetic resonance imaging data.

18. The system of claim 10, wherein the portion of the patient's anatomy comprises the patient's knee and femur.
